(19) United States
(12) Patent Application Publication
(10) Pub. No.: US 2020/0315817 A1
(43) Pub. Date: Oct. 8, 2020

(54) DEVICE FOR FIXING TWO ORTHOPEDIC COMPONENTS TO ONE ANOTHER

(71) Applicant: Otto Bock Healthcare Products GmbH, Wien (AT)

(72) Inventors: Robert KAITAN, (US); Robert HOFFMANN, (US); Alexander PAPPE, (US); Luis SAGMEISTER, (US); Thomas PAUSER, (US); Andreas SCHRAMEL, (US); Erik ALBRECHT-LAATSCH, (US); Michael NOLTE, (US); Andreas WEIGL-POLLACK, (US)

(73) Assignee: Otto Bock Healthcare Products GmbH, Wien (AT)

(21) Appl. No.: 15/733,267

(22) PCT Filed: Dec. 18, 2018
(86) PCT No.: PCT/EP2018/085661

§ 371 Date: Jun. 18, 2020

(30) Foreign Application Priority Data
Dec. 22, 2017 (DE) 10 2017 131 193.9

(51) Int. Cl. A61F 2/60; A61F 2/70; A61F 2/80

(52) U.S. Cl. CPC A61F 2/604; A61F 2002/5083; A61F 2/80; A61F 2/70

(57) ABSTRACT

A device for fixing two orthopedic components to one another includes a receiving element arranged on a first orthopedic component, which receiving element has a receptacle and includes a connecting element arranged on a second orthopedic component. The connecting element has a connector. The connector can be inserted into the receptacle and is coupled with the receiving element in a tensile force, compressive force, and torque-transmitting manner. At least one flow- and/or data-transmitting contact is arranged on the connecting element and the receiving element so as to correspond to one another, which form an electrical and/or data-transmitting connection with one another in a fixed state of the orthopedic components.

The invention relates to a device for fixing two orthopedic components to one another, comprising a receiving element arranged on a first orthopedic component, which receiving element has a receptacle, and comprising a connecting element arranged on a second orthopedic component, which connecting element has a connector which can be inserted into the receptacle and is coupled to the receiving element in such a way as to transmit tensile forces, compressive forces and torques.

An orthopedic system is composed of several orthopedic components assembled to form an orthopedic unit and has a functionality going beyond the functionality of the orthopedic components. An orthopedic system is, for example, an orthosis, a prosthesis or a wheelchair, and parts of such a system are considered to be orthopedic components, for example prosthetic or orthotic joints, tube adapters, prosthetic hands, rotary adapters, prosthesis sockets, orthosis rails, foot shells, hip shells, fastening devices for fastening orthoses to limbs, drives, data loggers, radio modules, feedback elements, energy stores, damper devices with hydraulic, magnetorheological and/or pneumatic dampers, drives, gears, sensors, and operating devices. An orthopedic system can be, for example, a thigh prosthesis with a prosthesis socket, a prosthetic knee joint and a lower-leg tube with a prosthetic foot arranged thereon, and a below-knee prosthesis can be composed of a lower leg socket with a prosthetic foot, if appropriate with a prosthetic ankle joint. If a thigh socket is connected to a lower-leg tube in an articulated manner via a prosthetic knee joint, a drive and/or a damper device can be arranged between the thigh socket and the lower-leg tube and can be controlled, for example, by a processor on the basis of sensor values. Depending on sensor data, drives are activated or deactivated or damper properties are changed, for example a flexion resistance is reduced or increased.

An analogous set-up can be provided in the case of orthoses. Wheelchairs can supplement orthotic and/or prosthetic management of patients or can constitute an independent orthopedic system.

Particularly in complex orthopedic systems, it is possible to provide several electrical devices such as electrical consumers, motors, actuating elements, sensors, processors, coils for changing the properties of magnetorheological liquids, or steering devices in the case of wheelchairs.

DE 19 22 619 B1 discloses an adjustable connecting element for prosthesis parts, consisting of a ball joint which can be locked in defined positions and on the spherical cap of which a preferably four-sided pyramid is placed with its tip pointing to the ball center point, which pyramid engages in a recess of the ball socket, in which set screws are arranged to bear on the side faces of the pyramid. This so-called pyramid adapter allows orthopedic components to move relative to one another by means of the two orthopedic components on the ball joint being moved relative to each other. In the desired position that is set, the position found is fixed by tightening the set screws. By means of the pyramid shape, the ball socket is braced relative to the spherical cap such that the two components are fixed relative to each other in all directions. Such a pyramid adapter has long been known for releasable and adjustable connection of prosthesis components relative to one another and is used as standard, for example for connecting a prosthesis socket to a prosthetic joint or for connecting a lower-leg tube to a prosthetic joint.

In the case of orthoses, rails or shells are connected to orthosis joints via so-called rail boxes, which have a recess within a support, in which recess a rail or a correspondingly configured projection can be inserted. By means of screws or clamping devices, the rail or the projection is locked in the rail box such that, similarly to the case of the pyramid adapter, it is possible for tensile forces, compressive forces and torques to be transmitted at the connection site.

By means of such devices for connecting several orthopedic components such as prosthesis parts or orthosis parts, it is possible to achieve a modular set-up of orthoses or prostheses and to adapt them easily to the respective patient.

Orthoses and prostheses have an increasingly complex set-up with drives, sensors and data processors. The object of the present invention is to simplify the assembly of the orthopedic components to form an orthopedic system.

According to the invention, this object is achieved by a device having the features of the main claim. Advantageous embodiments and developments of the invention are disclosed in the dependent claims, the description and the figures.

In the device for fixing two orthopedic components to one another, comprising a receiving element arranged on a first orthopedic component, which receiving element has a receptacle, and comprising a connecting element arranged on a second orthopedic component, which connecting element has a connector which can be inserted into the receptacle and is coupled to the receiving element in such a way as to transmit tensile forces, compressive forces and torques, provision is made that at least one contact, preferably several current-transmitting and/or data-transmitting contacts, is/are arranged on the connecting element and the receiving element in a corresponding manner, which contacts together form an electrical and/or data-transmitting connection when the orthopedic components are fixed to one another. By virtue of the arrangement or formation of at least one contact or contacts both in the connecting element and in the receiving element, it is possible to make available a device which, in addition to a mechanical connection of orthopedic components, at the same time produces an electrical connection, such that, for example, data can be transmitted via the contacts and/or electrical energy from an energy source, for example an accumulator, can be transmitted to a consumer, for example a drive. The electrical and/or data-transmitting contacts are produced automatically during mechanical coupling of the orthopedic components to one another, as a result of which it is possible to safely and permanently permit not only a mechanical connection but also an electrical and/or data-transmitting connection of orthopedic components such as orthosis parts or prosthesis parts. It is possible to arrange at least one contact on the connecting element and one contact on the receiving element. For the sake of simplicity, multiple contacts are discussed below, but this is not to be regarded as limiting the invention to a plurality of contacts.

The connector can be designed as a stub, or as a pyramid, truncated pyramid, truncated cone or cone with its tip pointing toward the connecting element, in order to permit and facilitate a form-fit connection of connecting element and receiving element to each other. By way of the connector, the connecting element can be adjusted, oriented and fixed relative to the receiving element, such that an adjustable orientation of the orthopedic components relative to one another can be obtained.

The connector can be provided with one or more spring-loaded or pretensioned locking elements, so as to be able to produce a simple and reversible plug connection between the connecting element and the receiving element. The locking element can be designed as a protection against withdrawal, as a clip or as an elastic projection, which fixes the two elements to each other after the connecting element has been joined to the receiving element.

The receiving element and the connecting element can have fastening elements with which it is possible to fix the receiving element and the connecting element reversibly on the respective orthopedic component. Alternatively, provision is made that the receiving element or the connecting element is designed as an integral constituent part of the respective orthopedic component.

To permit simplified joining of the receiving element to the connecting element, the receptacle in the receiving element is designed corresponding to the connector. If a subsequent adjustment is to be carried out after the joining procedure, an embodiment of the receptacle is provided which permits a movement of the receiving element relative to the connecting element before the desired position is finally fixed in each case.

To allow the receiving element and connecting element and therefore the orthopedic components to be fixed relative to one another adjustably in the desired position, the receiving element has at least one tensioning element, preferably several tensioning elements, which is/are movable from an insertion position, in which the connector is insertable into the receptacle, to a locking position, in which the connector is fixed in the receptacle. These tensioning elements can be designed in particular as set screws, toggles, latching pins or wedges. The tensioning elements are arranged and designed such that a connection between the receiving element and the connecting element is produced via which tensile forces, compressive forces and torques can be transmitted. By way of the connecting element and the receiving element, the mechanical forces passed through the two orthopedic components are transmitted permanently and safely. The tensioning elements preferably act transversely or at an angle of between 80° and 90° to the contact surface between the tensioning element and the connector. The tensioning elements can also penetrate into the connector and can there produce a form-fit lock against a relative displacement of connecting element and receiving element. For example, pins, stubs or screws can be inserted into the connector.

In a variant of the invention, provision is made that the locking element and the receiving element form a ball joint, wherein a socket is formed on the receiving element and a spherical cap is formed on the connecting element, such that angle adjustability of receiving element and connecting element relative to each other is provided in all directions of rotation. Depending on the design of the connector, which is mounted on the spherical cap, it is also possible to permit a free rotation about the longitudinal extent of the connector. Depending on the design of the receptacle, it is possible, even with a straight-wall configuration of the connector, to obtain any desired rotation of the connecting element relative to the receiving element about an axis parallel to the longitudinal extent of the connector, until fixing via tensioning elements, e.g. set screws, takes place. In addition to rotational degrees of freedom, the coupling of the connector in the receptacle also permits a displaceability of the components before the desired position is fixed.

The contacts can be arranged or formed on surface regions of the connecting element and of the receiving element. The connecting element and the receiving element are preferably made of metal, in particular light metal. The contacts are arranged or formed on surface regions electrically insulated from one another in particular, such that electrical contact or data-transmitting contact takes place at several contact faces together with the connection between the connecting element and the receiving element. Conversely, the separation of the electrical or data-transmitting contacts is effected when the mechanical connection between the connecting element and the receiving element is undone. The plurality of electrical and/or data-transmitting contacts, which are separate from each other, are preferably arranged or formed on the end faces and side faces of the connector. Likewise, they can be arranged or formed on other abutting surfaces of the connecting element and of the receiving element, for example on the socket and the spherical cap in the case where the connecting element and the receiving element are configured as a ball joint. The contacts are then configured as surface contacts which produce the desired connection when pressed onto each other.

The contacts can be designed as plug contacts which, together with the connection of connecting element and receiving element, form a plug contact with each other.

The contacts can be arranged in a plug and a bushing, which are arranged on or in the connecting element and the receiving element. It is thereby possible to manufacture the plug contacts separately from the receiving element and the connecting element and subsequently to connect them to or integrate them with the connecting element and the receiving element in an assembly procedure. The electrical and/or data-transmitting contacts can then be connected via lines or cables to further electrical or data-processing devices in the orthopedic system or the orthopedic component. The plugs can be mounted in a floating fashion on or in the connecting element and the receiving element in order to tolerate assembly play and, in the event of an adjustment of the connecting element relative to the receiving element, to further ensure safe plug contact. The plug and the bushing can be connected to each other via a magnetic coupling, so as to provide independent contacting and additional safeguarding of the contact for transmission of energy and data.

The delivery of the energy or data to the contacts preferably takes place via cables which are flexible in length and/or are guided in a link chain. A line that is adjustable in length is, for example, a spirally wound line with an elastic sheath, which particularly facilitates assembly. If plug contacting is provided in which the plug and/or the bushing are/is mounted in a floating fashion, a length-adjustable connection makes available the sufficient line length for reliably producing the contacts. After assembly, the line is under slight tensile stress, such that the respective line remains held in place. The plug contact is maintained, for example, by frictional forces or a magnetic coupling. The tension in the line prevents a situation where the line is arranged loosely within the orthopedic component in the assembled state. The guiding in a link chain is particularly expedient if one of the orthopedic components is a joint, so that a relative displacement of orthopedic components relative to each other is intended to take place.

In a development of the invention, provision is made that at least one passage through which a line passes from a first component to a second component is formed in the receiving element and in the connecting element. Through this passage, it is possible, for example, to guide cables or hydraulic or pneumatic lines from a first component to a second component through the connection site between the two components. Through this passage, a line can be routed inside the components, which leads to improved protection against buckling and to improved protection against external influences. The passage is preferably formed by recesses and/or bores inside the connecting element and the receiving element. In an embodiment of the connecting element with a connector, for example a pyramid connector or pyramid adapter, and with the receiving element as a tube adapter for receiving the connector or pyramid adapter, bores are formed flush with one another in the tube adapter and in the pyramid adapter, such that, after assembly, a free passage from a distal component to a proximal component is possible. The respective bore is then continued, for example through a tube attachment or a tube portion integrally formed or secured on the tube adapter, and through the connecting element, for example a pad for receiving the pyramid adapter. A passage in the receiving element and in the connecting element, for routing a line from a first component to a second component inside the receiving element and the connecting element, can also be configured without energy-transmitting and/or data-transmitting contacts on the connecting element and the receiving element. The passage of lines such as cables or hydraulic elements can be provided as an independent solution.

Illustrative embodiments of the invention are explained in more detail below with reference to the accompanying figures, in which:

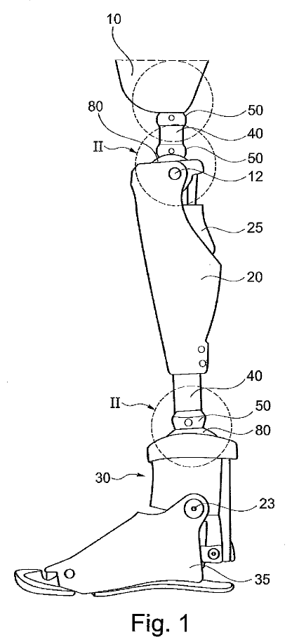

FIG. 1 shows a prosthetic leg;

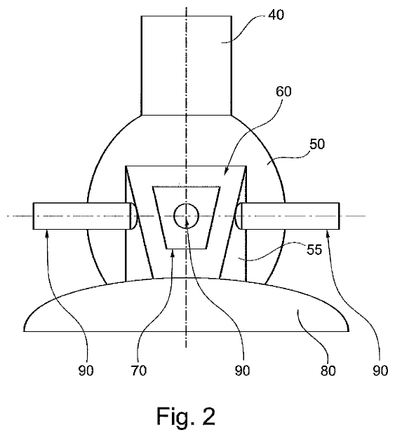

FIG. 2 shows a schematic sectional view through a receiving element and a connecting element according to detail II;

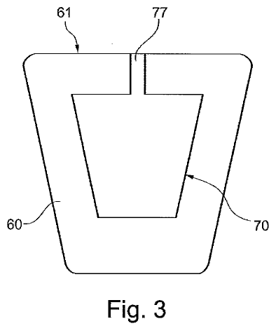

FIG. 3 shows a view of a connecting element on its own, with electrical contacting;

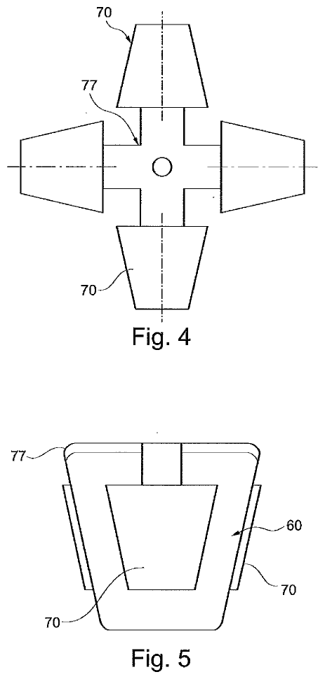

FIG. 4 shows a view of an electrical connecting element on its own;

FIG. 5 shows an assembly variant of the electrical connecting element according to FIG. 4;

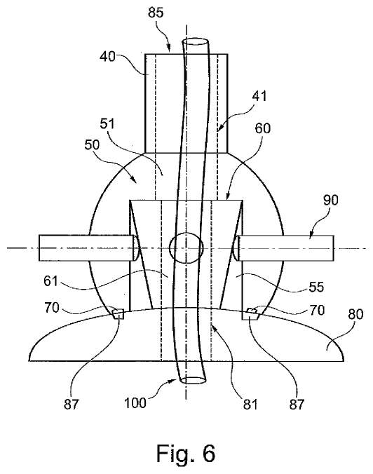

FIG. 6 shows a variant according to FIG. 2 without electrical contacting and with a passage, and

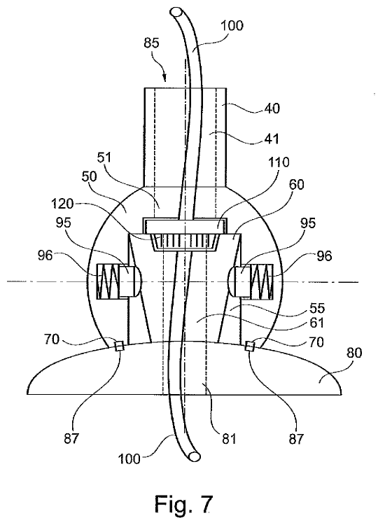

FIG. 7 shows a variant of FIG. 6 with a plug connection.

FIG. 1 shows a schematic view of a prosthesis of a lower extremity, in the form of a prosthetic leg with several orthopedic components 10, 20, 30, which are connected to one another. The first orthopedic component 10 is a thigh socket, on which a tube portion 40 is secured via a receiving element 50, which is explained in more detail below. At the end of the tube portion 40 directed away from the thigh socket 10, there is a second receiving element 50, which interacts with a connecting element 80, explained in more detail below, in order to connect the first component 10 to a second component 20 in the form of a below-knee part, specifically with form-fit engagement and in a manner that transmits forces and torques. The below-knee part 20 is coupled to the thigh socket 10 in such a way as to be pivotable about a pivot axis 12. In the illustrative embodiment shown, the below-knee part 20 accommodates a hydraulic actuator 25, which has electrical and/or electronic devices. The hydraulic actuator 25 can be designed as a passive component and have a hydraulic damper comprising servo controls via which valves are opened and closed, in order to be able to adjust an extension resistance and/or flexion resistance. In an embodiment of the hydraulic actuator 25 as a drive, a pump device or a mechanical energy store, for example in the form of a spring or a pressure accumulator, can be assigned to the hydraulic actuator 25, such that it is possible to effect or at least support a movement of the thigh socket 10 relative to the below-knee part 20. Sensors, computing devices, control devices and devices for receiving data or for transmitting data and energy can likewise be provided in the below-knee part 20.

In the distal region of the below-knee part 20, a further tube portion 40 is provided, at the distal end of which a third receiving element 50 is arranged via which, in interaction with a further connecting element 80, a connection to a prosthetic ankle joint 30 is produced. By way of the prosthetic ankle joint 30, it is possible to pivot a foot part 35, which is arranged on the prosthetic ankle joint 30 about a pivot axis 23. The prosthetic ankle joint 30 can be designed as an electrical and/or hydraulic actuator and can damp or support or bring about a plantar flexion and/or dorsal flexion of the foot part 35. For this purpose, storage devices for electrical and/or mechanical energy can be arranged in the prosthetic ankle joint 30 and can be activated or deactivated via a control device and corresponding actuators. The activation or deactivation advantageously takes place on the basis of sensor-based control.

Screws, in particular grub screws, are arranged inside the receiving elements 50 and permit mechanical locking with the connecting element 80. In the illustrative embodiment shown, three orthopedic components 10, 20, 30 are present, wherein the first component 10 is connected to the below-knee part, as second component 20, via the receiving element 50 on the tube portion and the connecting element 80 is connected to the upper part of the prosthetic knee joint. The below-knee part 20 forms the first component for the connection to the prosthetic ankle joint 30, while the prosthetic ankle joint 30 forms the second component. In principle, it is also possible that the receiving element 50 is arranged on the distal component of the component connection, while the connecting element 80 is arranged on the proximal component.

FIG. 2 shows a sectional view according to detail II in FIG. 1. The tube portion 40 is connected to the receiving element 50 in the form of a tube adapter or is formed in one piece therewith. The receiving element 50 is preferably made of a rigid metal, in particular a light metal such as titanium or an aluminum alloy. Other materials are also possible in principle, for example steel, plastics or fiber-reinforced composites. Formed inside the receiving element 50 is a receptacle 55 in the form of a hollow space into which lateral bores open. These bores are provided with threads via which screws 90 can be screwed in radially from the outside. In the illustrative embodiment shown, the receiving element 50 is provided with a round cross section, while the receptacle 55 has, for example, a square or round cross section.

A connector 60 in the form of a pyramid adapter is inserted inside the receptacle 55. The connector 60 has a shape like a pyramid or cone, wherein the tip of the pyramid or of the cone points in a direction away from the tube 40, in the distal direction in the illustrative embodiment shown. The region of the pyramid tip or cone tip is adjoined by the connecting element 80, which has a surface like a spherical cap, such that in a released state, when the screws 90 are not tightened, the connecting element 80 can be moved relative to the receiving element 50. For this purpose, the connector 60 is slightly smaller than the receptacle 55, such that a pivoting and twisting of the orthopedic components 10, 20 can take place via the connection to the receiving element 50 and to the connecting element 80. After a corresponding pivoting or twisting and aligning of the individual components 10, 20, 30 with respect to one another, the screws 90 are tightened until they bear on the connector 60. By way of the oblique shape of the side surfaces of the connector, the receiving element 50 is pressed in the direction of the connecting element 80 and produces a stable connection that transmits force and torque. The connector 60 can be integrally formed on the connecting element 80 or can be secured thereto. The securing can be reversible and involve form-fit engagement or cohesive bonding, for example by welding.

Energy-transmitting and data-transmitting contact faces 70 are arranged or formed on the outside of the connector 60 and, by way of cables or conducting tracks inside the second component, allow data and/or electrical energy to be forwarded via the connecting element 80. The contact faces 70 can be arranged or formed on all sides of the connector 60, wherein the connector 60 is either non-conductive itself or has an insulating coating, such that a short circuit does not occur or the data line is not interrupted. Forwarding from the contact faces 70 takes place via the screws 90, which are designed as conductive screws and at the same time form the tensioning elements with which the connector 60 is fixed in the receptacle 55. The screws 90 as tensioning elements can be unscrewed such that the connector 60 is located in an insertion position. After the connector 60 has been inserted into the receptacle 50, the tensioning elements 90 formed as screws are screwed in and brought to a locking position (shown in FIG. 2) in which the connector 60 is fixed with form-fit engagement.

In an embodiment of the connector 60 as a four-sided pyramid adapter, four contact faces 70 are present, such that data and/or energies can be transmitted via four conductive screws 90, which also from contacts. The screws 90 are electrically insulated from the tube adapter 50 and are connected to current lines and/or data lines, in order to permit and produce a corresponding connection of the upper part or the first component 10 to the second component 20.

FIG. 3 shows a view of a connector 60 on its own. The connector 60 is designed as a pyramid adapter and is made of a non-conductive material or is provided with an insulating coating. The contact face 70 on an outer side is continued via an electrical connection element 77 to an end region 61 of the connector 60. By way of contacting at the end face, it is possible to achieve electrical contacting on a corresponding mating face inside the receptacle 55 in order to transmit data and/or energy. For this purpose, a corresponding contact is arranged or formed in the bottom face of the receptacle 55. Thus, in addition to or instead of electrical contacting via the conductive screws 90, contacting can take place via the end faces or bottom faces of the connector 60 and of the receptacle 55. The forwarding of energy or data then takes place through the receiving element 50 in the form of the tube adapter via insulated lines, contact regions or the like.

FIG. 4 shows a variant of an embodiment of a contact field with a multiplicity of electrical contacts 70. For use on a four-sided connector, four contacts 70 or contact plates 70 are provided which are arranged in a star shape. The individual contacts 70 are mechanically connected to one another via a cross-shaped electrical connecting element 77. The electrical connecting element 77 can insulate individual regions from one another, such that contacts 70 are not electrically coupled to one another. It is likewise possible that, for example, two contacts 70 are electrically connected to each other via the electrical connecting element 77, such that, for example, electrical energy can be transmitted via two mutually opposite contacts 70 and data can be transmitted via the two other contacts. For assembly, the electrical connecting element is placed with the central recess or the central bore onto the end face 61 of the connector 60, and the contacts 70 are placed on the side walls of the connector 60. The electrical connecting element 77 can be plastically deformable. Alternatively or in addition, a securing device, for example an adhesive or the like, can be arranged on the contacts 70, in order to hold the contacts 70 on the side walls of the connector 60. The assembled state of the contacts 70 with the connecting element 77 on the connector 60 is shown in FIG. 5.

FIG. 6 shows a further variant of the invention, in which the mechanical set-up corresponds to that of FIG. 2. Here too, a tube portion 40 is connected to a receiving element 50 or is formed in one piece therewith. By way of screws 90, designed in the illustrative embodiment as grub screws, a connector 60 in the form of a pyramid adapter is mechanically fixed inside a recess 55 of the receiving element 50. The connector 60 is secured to the connecting element 80 or is formed thereon. In the tube piece 40 and also in the receiving element 50, bores 41, 51 are formed, which are oriented corresponding to the bores 81, 61 in the connector 60 and the connecting element 80. By means of the bores 41, 51, 61, 81 lying flush with one another, it is possible to permit a passage 85 from the first component 10 to the second component 20, such that a data or energy cable 100 or another line can be routed from the first component 10 to the second component 20. The routing of the line or of the cable 100 can also take place from the second component 20 to the third component 30, for example in order to capture sensor data, drive motors or adjust valves. No electrical contacts need be arranged on the outsides of the pyramid adapter or connector 60, although they can be thus arranged. It is likewise possible that electrical contacts 70 are arranged on the end faces of the receiving element 50 and are arranged corresponding to contact regions 87 that are arranged on end faces of the spherical cap-shaped surface of the connecting element 80. It is thereby possible, by placing the receiving element 50 on the connecting element 80, to establish an electrical and/or data-transmitting connection from the first component 10 to the second component 20. Mechanical fixing is then provided by the screws 90 and the connector 60 in the receptacle 55.

A combination of the embodiments of FIGS. 2 and 6 is likewise possible and is a subject of the invention, such that contacts 70 on the side walls of the connector 60 and also on the end faces of the receiving element 50 and of the connecting element 80 are present via contacts 70 and/or contact regions 87, and contacting takes place at a large number of contact sites.

FIG. 7 shows a further variant of the invention which, in terms of its set-up, corresponds to the embodiment according to FIG. 6. Instead of the radially inwardly directed screws 90 which fix the connector inside the recess 55, the connector 60 in the illustrative embodiment according to FIG. 7 is fixed in the recess 55 via several locking elements 95, which are pretensioned by a spring element 96 in the direction of the connector 60. The locking elements 95 are pressed against the outer wall of the connector 60 by the elastic pretensioning provided by the springs 96, which are designed as helical springs or as elastomer elements or can also be constructed in some other shape. The locking element 95 and also the spring 96 can be electrically conductive and can be used instead of the screws in the illustrative embodiment according to FIG. 6. The oblique position of the outer wall of the connector 60 results in a force component via which the receiving element 50 is pressed in the direction of the connecting element 80. In the passage 85, a cable or a line 100 is guided through the bore 41 and has, at its end, a plug 110 in which at least one contact is arranged for transmitting electrical energy and/or data. The plug 110 is arranged in a recess in the bottom face of the receptacle 55 and, if appropriate, is fixed there. On the opposite end face of the connector 60, a depression is arranged in which a bushing 120, with corresponding contacts or receptacles for the contacts of the plug 110, is arranged and fixed. From the bushing 120, a line 100 runs through the bore 81 to a further electrical and/or electronic component. The plug 110 and the bushing 120 are arranged such that they produce an electrical and/or data-transmitting connection when the receiving element 50 is connected to the connecting element 80 and if appropriate when the connector 60 is inserted fully into the receptacle 55. The cables 100 serve as a line for the electrical and data-transmitting connection between several electrical and/or electronic devices.

1. A device for fixing two orthopedic components to one another, comprising:
a receiving element arranged on a first orthopedic component, which receiving element has a receptacle;
a connecting element arranged on a second orthopedic component, which connecting element has a connector which can be inserted into the receptacle and is coupled to the receiving element in such a way as to transmit tensile forces, compressive forces and torques;
at least one of a current-transmitting contact and a data-transmitting contact arranged on the connecting element and the receiving element in a corresponding manner, the at least one contact forms at least one of an electrical connection and a data-transmitting connection when the orthopedic components are fixed to one another.

2. The device as claimed in claim 1, wherein the connector is designed as a stub, or as a pyramid or cone with its tip pointing toward the connecting element.

3. The device as claimed in claim 1, wherein the connector has at least one spring-loaded or pretensioned locking element.

4. The device as claimed in claim 1, wherein the receptacle is designed corresponding to the connector.

5. The device as claimed in claim 1, wherein the receiving element has at least one tensioning element which is movable from an insertion position, in which the connector is insertable into the receptacle, to a locking position, in which the connector is fixed in the receptacle.

6. The device as claimed in claim 1, wherein the connecting element and the receiving element form a ball joint with a socket and a spherical cap.

7. The device as claimed in claim 1, wherein the at least one contact is arranged or formed on surface regions of the connecting element and of the receiving element.

8. The device as claimed in claim 1, wherein the at least one contact is designed as a plug contact.

9. The device as claimed in claim 1, wherein the at least one contact is arranged in a plug and a bushing which are arranged on or in the connecting element and the receiving element.

10. The device as claimed in claim 1, wherein the at least one contact is connected by cables to at least one electronic device.

11. The device as claimed in claim 10, wherein the cables are at least one of flexible in length and guided in a link chain.

12. The device as claimed in claim 1, wherein a passage through which a line passes from the first orthopedic component to the second orthopedic component is formed in the receiving element and the connecting element.

13. The device as claimed in claim 12, wherein the passage is formed by two mutually corresponding bores or recesses in the receiving element and the connecting element.

14. A device for fixing a first orthopedic component to a second orthopedic component, comprising:
a receiving element arranged on the first orthopedic component and having a receptacle;
a connecting element arranged on the second orthopedic component and having a connector, the connector being insertable into the receptacle and coupled to the receiving element to transmit tensile forces, compressive forces and torques;
at least one current-transmitting contact or data-transmitting contact arranged on the connecting element and the receiving element, the at least one current-transmitting contact or data-transmitting contact forming an electrical connection or data-transmitting connection when the orthopedic components are fixed to one another.

15. The device as claimed in claim 14, wherein the connector is designed as a stub, or as a pyramid or cone with a tip pointing toward the connecting element.

16. The device as claimed in claim 14, wherein the connector has at least one spring-loaded or pretensioned locking element.

17. The device as claimed in claim 14, wherein the receptacle is designed to match a shape of a portion of the connector.

18. The device as claimed in claim 14, wherein the receiving element has at least one tensioning element, the at least one tensioning element being movable from an insertion position in which the connector is insertable into the receptacle, to a locking position in which the connector is fixed in the receptacle.

19. The device as claimed in claim 14, wherein the connecting element and the receiving element form a ball joint with a socket and a spherical cap.

20. The device as claimed in claim 14, wherein the at least one current-transmitting contact or data-transmitting contact is arranged or formed on surface regions of the connecting element and of the receiving element.
